(19) United States
(12) Patent Application Publication
(10) Pub. No.: US 2023/0392179 A1
(43) Pub. Date: Dec. 7, 2023

(54) SCREENING SYSTEM FOR MYCOBACTERIUM TUBERCULOSIS INTEIN SPLICING INHIBITOR, CONSTRUCTION METHOD AND USE THEREOF

(71) Applicant: SOOCHOW UNIVERSITY, Suzhou (CN)

(72) Inventors: Xingmei QI, Suzhou (CN); Sidong XIONG, Suzhou (CN); Yiyang LI, Suzhou (CN); Jialan ZHANG, Suzhou (CN)

(21) Appl. No.: 17/912,466

(22) PCT Filed: Jul. 16, 2021
(86) PCT No.: PCT/CN2021/106637

§ 371 Date: Jan. 5, 2023

(30) Foreign Application Priority Data
Jul. 1, 2021 (CN) 202110749575.3

(51) Int. Cl. C12Q 1/02; C12N 15/74; C07K 14/35

(52) U.S. Cl. CPC C12Q 1/025; C12N 15/74; C07K 14/35; C12N 2830/42

(57) ABSTRACT

The present invention provides a Mycobacterium tuberculosis intein splicing inhibitor screening system, where Mycobacterium smegmatis is used as a model bacterium, a gene sequence of a protein regulated by an intein is inserted into a 65S site of a kanamycin-resistant protein gene, a vector with first antibiotic resistance is used for constructing a recombinant plasmid, and the recombinant plasmid is transferred into Mycobacterium smegmatis and is cultured in a medium containing a first antibiotic and kanamycin, to obtain the Mycobacterium tuberculosis intein splicing inhibitor screening system. The first antibiotic is any antibiotic other than kanamycin. By inhibiting the splicing activity of inteins to influence the activity of kanamycin-resistant protein, the growth of the recombinant Mycobacterium smegmatis in a medium containing kanamycin is influenced, so as to screen for drugs with an inhibitory effect on the inteins, thereby achieving simple, efficient, and rapid screening of new anti-tuberculosis drugs.

### FIELD OF THE INVENTION

The present invention relates to the field of biotechnology, and more particularly to a Mycobacterium tuberculosis intein splicing inhibitor screening system, a construction method and use thereof.

### DESCRIPTION OF THE RELATED ART

In recent years, the co-infection of tuberculosis and AIDS and the problem of multidrug-resistant tuberculosis have become more and more serious, posing great challenges to the clinical treatment of tuberculosis. Therefore, the development of new anti-tuberculosis drugs has become an urgent task in the prevention and treatment of tuberculosis. Three important enzymes RecA, DnaB, and SufB in Mycobacterium tuberculosis (MTB) are regulated by inteins, respectively, and must complete post-translational protein splicing in order to function. They play an important role in the growth and reproduction of MTB. According to the different inserted host genes, the three inteins contained in MTB are named Mtu RecA, Mtu DnaB, and Mtu SufB respectively. Since the amino acids at the active center of intein splicing are highly conserved, inhibiting the activity of any one of the inteins may also affect the others. Therefore, intein-targeted protein splicing inhibitors can become new targets of anti-tuberculosis drugs and can minimize the occurrence of drug-resistant mutants. An intein is an insertion sequence that exists in a precursor protein. During the post-translational processing and maturation of the protein, the intein self-cleaves from the precursor protein, and exteins on both sides are linked via a natural peptide bond to form a mature functional protein. This process is known as protein splicing. Protein splicing occurs only in bacteria, archaea, and unicellular eukaryotic cells, but not in higher eukaryotes, so inhibitors that specifically target inteins are biologically safe for humans. As MTB is highly infectious (BSL-3) and has a long growth cycle (100 days), the direct application of MTB to screen new anti-tuberculosis drugs targeting inteins is greatly limited. Intein inhibitor screening systems that have been reported include in-vitro screening systems that rely on fluorescent proteins and in-vivo screening systems that rely on bacterial thymidylate synthase, bacterial CcdB toxin, and bacterial DNA helicase subunit A, but such in-vitro screening systems require the purification of the protein for in-vitro screening, which is complicated, time-consuming, and labor-intensive. Other screening systems all use Escherichia coli as the host, and the screening results are quite different. In addition, for reported screening systems, the splicing activity of inteins is low, resulting in low screening sensitivity. Therefore, it is necessary to establish a simple and reliable screening system for intein inhibitors.

### SUMMARY OF THE INVENTION

In order to solve the above technical problems, the present invention provides a Mycobacterium tuberculosis intein splicing inhibitor screening system, which inhibits the splicing activity of inteins to influence the activity of kanamycin-resistant protein, so as to influence the growth of Mycobacterium smegmatis. Drugs with an inhibitory effect on the inteins are screened for through observation of the growth of Mycobacterium smegmatis, thereby achieving simple, efficient, and rapid screening of splicing inhibitors or new anti-tuberculosis drugs.

In the Mycobacterium tuberculosis intein splicing inhibitor screening system of the present invention, Mycobacterium smegmatis is used as a model bacterium, a gene sequence of a protein regulated by an intein is inserted into a 65S site of a kanamycin-resistant protein gene, a vector with first antibiotic resistance is used for constructing a recombinant plasmid, and the recombinant plasmid is transferred into Mycobacterium smegmatis and is cultured in a medium containing a first antibiotic and kanamycin, to obtain the Mycobacterium tuberculosis intein splicing inhibitor screening system. The protein regulated by the intein is selected from a full-length or mini-fragment of Mycobacterium tuberculosis RecA intein, DnaB intein, or SufB intein. The first antibiotic is any antibiotic other than kanamycin.

In the present invention, the mini-fragment (mini type) refers to the removal of the homing endonuclease activity of the full-length intein to retain only the splicing activity region.

Mycobacterium smegmatis (Msm) used in the present invention has a high similarity to Mycobacterium tuberculosis genes, and has the advantages of fast growth (3-5 days), low infectivity (BSL-1), and no intein sequence in the bacterial genome. Therefore, Mycobacterium smegmatis is used as a model organism, and an intein splicing inhibitor screening system relying on kanamycin resistance is introduced exogenously to simulate Mycobacterium tuberculosis to establish an in-vivo screening system for the rapid screening of splicing inhibitors or anti-tuberculosis drugs. After an intein inhibitor is added to the medium of the screening system, the splicing activity of inteins is inhibited, and the activity of the kanamycin-resistant protein cannot be recovered, so Mycobacterium smegmatis will not be able to grow in the medium containing kanamycin.

Compared to the problems of false positives or missed selections caused by the low sensitivity and specificity of the screening results in the existing screening systems using Escherichia coli as the model bacteria, the innovations of the present invention are as follows: (1) The splicing activity of inteins is greatly affected by the host bacteria, and the splicing sites in model bacteria such as Escherichia coli may not be applicable to Mycobacterium smegmatis. In the present invention, the insertion of the intein gene sequence into the kanamycin-resistant protein not only needs to ensure that the activity of the kanamycin-resistant protein is interrupted, but also needs to ensure that the intein has certain splicing activity at this site, to allow for the occurrence of protein splicing to reconstruct the activity of the kanamycin-resistant protein. It is found by the inventors through tests on different sites that the 65S site has a better effect and the highest splicing activity. (2) The results of the respective insertion of the full-length and mini-fragment (mini type) of Rec A intein in a screening system using Escherichia coli as the model bacteria show that the splicing efficiency of the full-length type is about 30%, while almost no splicing activity is observed for the mini type. In practical applications, a mini type intein is constructed by removing the homing endonuclease activity of the full-length intein to retain only the splicing activity region, which can greatly shorten the intein sequence, thereby facilitating the construction and expression of the gene. (3) In the screening system of the present invention, the mini type of Rec A intein is inserted at the 65S site, and the splicing efficiency of the mini type is greatly improved, which can be up to about 90%. The improvement of the splicing efficiency is conducive to improving the sensitivity of the screening system, indicating that the intein shows a higher activity in Mycobacterium smegmatis. Therefore, the screening system using Mycobacterium smegmatis as the host can give more reliable screening results, thereby achieving the screening of new anti-tuberculosis drugs with specific inhibitory effects on inteins.

Preferably, the vector used in the present invention includes but is not limited to a PMV261 vector.

A method for constructing the screening system of the present invention includes the following steps:
(1) inserting a gene sequence of a protein regulated by intein into a 65S site of a kanamycin-resistant protein gene to construct a fusion gene;
(2) constructing the fusion gene in the step (1) in a vector with first antibiotic resistance to obtain a recombinant plasmid;
(3) transferring the recombinant plasmid in the step (2) into Mycobacterium smegmatis to obtain recombinant Mycobacterium smegmatis; and
(4) inoculating the recombinant Mycobacterium smegmatis in the step (3) in a medium containing a first antibiotic and kanamycin, to obtain the Mycobacterium tuberculosis intein splicing inhibitor screening system.

Preferably, in the step (2), cleavage sites of the fusion gene and the vector are both BamHI and HindIII.

Preferably, in the step (4), the concentration of kanamycin in the medium is 30-50 μg/mL, more preferably 50 μg/mL. Concentrations in this range can inhibit the splicing activity of inteins. A too low concentration will lead to insensitive screening, and a too high concentration will affect the growth of Mycobacterium smegmatis.

Preferably, in the step (4), the concentration of the first antibiotic in the medium is 40-60 μg/mL.

The present invention provides use of the screening system in screening Mycobacterium tuberculosis intein splicing inhibitors, including the following steps: adding a Mycobacterium tuberculosis intein splicing inhibitor to the screening system, and carrying out high-throughput screening of the Mycobacterium smegmatis intein splicing inhibitor according to the growth of the recombinant Mycobacterium smegmatis.

Preferably, the concentration of the Mycobacterium tuberculosis intein splicing inhibitor is 1-100 μM.

Preferably, the use further includes culturing for 90-110 h after adding a Mycobacterium tuberculosis intein splicing inhibitor to the screening system. Mycobacterium smegmatis is grown to a suitable concentration for the detection of the OD value.

Preferably, an inhibitor capable of inhibiting the splicing activity of inteins is determined by detecting the OD value of the bacterial solution.

The present invention also provides use of the screening system in screening anti-tuberculosis drugs.

By virtue of the above solution, the present invention has at least the following advantages.
(1) The present invention uses Mycobacterium tuberculosis as a model bacterium to establish a screening system relying on kanamycin resistance, which, when used with appropriate splicing sites, shows high splicing activity for both full-length and mini type inteins, breaking through the limitations of no splicing activity or low splicing activity of the existing screening systems for mini type intein, thereby improving the sensitivity and accuracy of the screening system.
(2) The present invention proposes a screening system for intein inhibitors, and this strategy can be applied to the screening of other substances or drugs.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

To make the content of the present invention more comprehensible, the present invention will be described in further detail below according to specific embodiments of the present invention and in conjunction with the accompanying drawings, wherein:

FIG. 1 is a schematic diagram showing the construction of plasmid PMV261-Kana^{R} and PMV261-Kana^{R}-65S-RecA intein.

FIG. 2 shows the OD₆₀₀ of the bacterial solution of Mycobacterium smegmatis containing the plasmid PMV261-Kana^{R}-65S-RecA intein at different kanamycin concentrations and the effect of addition of 20 μM cisplatin on the growth of Mycobacterium smegmatis.

FIG. 3 shows the effect of addition of different concentrations of cisplatin to 50 μg/mL Kana on the growth of Mycobacterium smegmatis respectively containing plasmids PMV261-Kana^{R}-65S-RecA intein and PMV261-Kana^{R}.

FIG. 4 shows the result of Western blot of the intein splicing efficiency in Mycobacterium smegmatis containing plasmid PMV261-Kana^{R}-65S-RecA intein at Kana concentrations of 25 μg/mL and 50 μg/mL respectively.

### DETAILED DESCRIPTION OF THE PREFERRED EMBODIMENTS

The present invention will be further described below in conjunction with the accompanying drawings and specific embodiments, so that those skilled in the art can better understand and implement the present invention, but the embodiments described are not intended to limit the present invention.

### Example 1

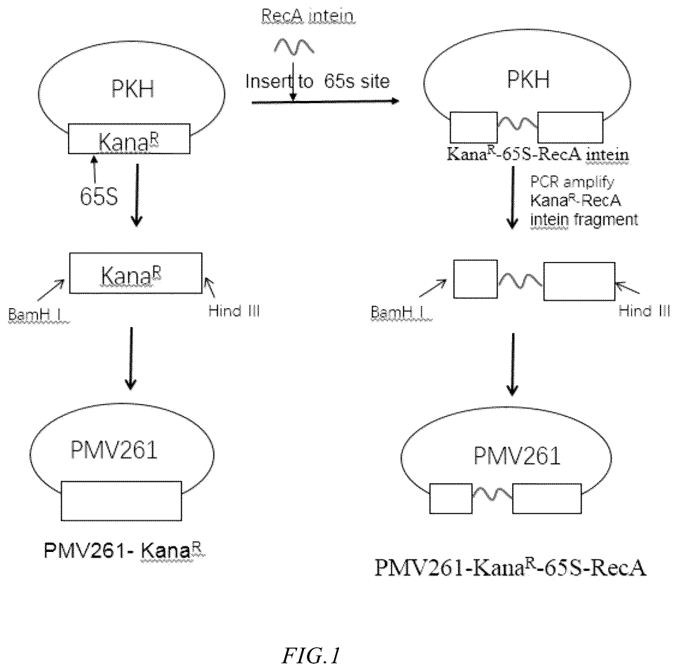

PKH-Kana^{R} and PKH-Kana^{R}-65S-RecAmini-intein plasmids were respectively used as templates to amplify a kana resistant gene (Kana^{R}) and a Kana^{R}-RecA mini-intein fusion gene. HindIII and BamHI cleavage sites were added on both sides of the primers. The PCR product was double digested by HindIII and BamHI, and then ligated to a vector PMV261 that had been digested by the same enzymes to construct plasmid PMV261-Kana^{R} and plasmid PMV261-Kana^{R}-65S-RecA intein (as shown in FIG. 1). The plasmids were electroporated respectively in Mycobacterium smegmatis, and positive clones were screened by hygromycin.

### Example 2

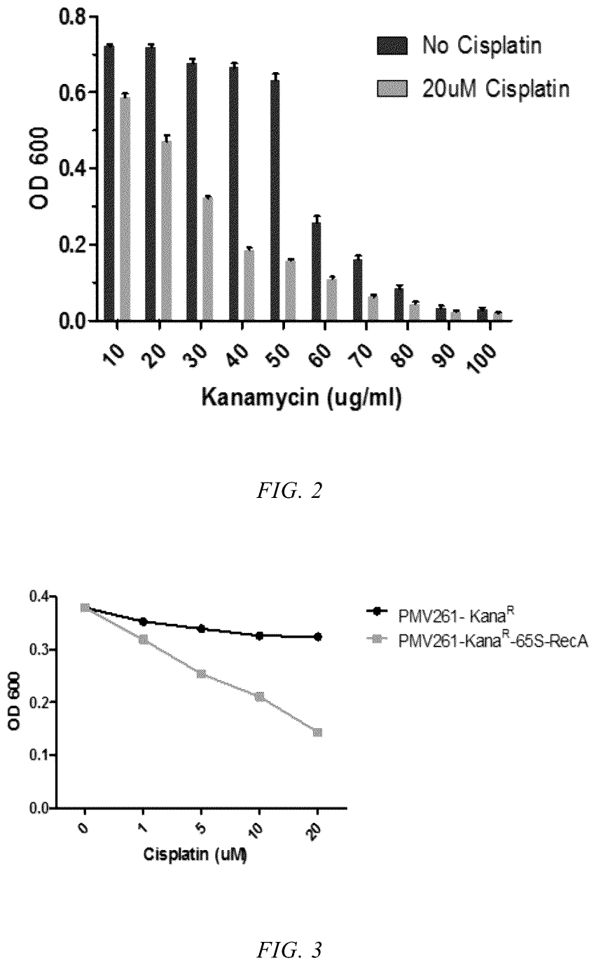

The positive clone containing the plasmid PMV261-Kana^{R}-65S-RecA intein was picked up and transferred to and cultured in a medium with hygromycin resistance, and allowed to stand in a special incubator at 37° C. for 3-4 days. When the OD value of the bacterial solution reached about 0.8, the bacterial solution is transferred to 5 mL of fresh liquid mediums containing different kana concentrations (10-100 μg/mL) at a ratio of 1:100. The OD value was detected after culturing for 100 h, and the effect of different kanamycin concentrations on the growth of Mycobacterium smegmatis was obtained (see FIG. 2, No Cisplatin). At the same time, the effect of addition of cisplatin in a final concentration of 20 μM on the growth of Mycobacterium smegmatis at different kanamycin concentrations (10-100 μg/mL) was tested (see FIG. 2, 20 μM Cisplatin).

The results in FIG. 2 show that a kanamycin concentration lower than 50 μg/mL has a small effect on the growth of Mycobacterium smegmatis, and a kanamycin concentration higher than 50 μg/mL has a great effect on the growth of Mycobacterium smegmatis. After the addition of 20 μM cisplatin, the inhibitory effect gradually increased with the increase of kanamycin concentration, so the kanamycin concentration in the subsequent screening system is preferably 50 μg/mL.

### Example 3

In order to prove that the inhibitory effect of cisplatin on the growth of Mycobacterium smegmatis is specific to intein, Mycobacterium smegmatis containing plasmid PMV261-Kana^{R} was used as the control group to test the effect of different concentrations of cisplatin on the growth at the kanamycin concentration of 50 μg/mL. The cultured Mycobacterium smegmatis containing PMV261-Kana^{R} and the cultured Mycobacterium smegmatis containing PMV261-Kana^{R}-65S-RecA intein were respectively transferred to 5 mL of medium containing two antibiotics Hyg+ and kana+ (50 μg/mL) at a ratio of 1:100, and different concentrations of cisplatin (0-20 μM) were added respectively. After the bacterial solution was allowed to stand in an incubator at 37° C. for 100 h, the OD₆₀₀ value of the bacterial solution was detected to verify the feasibility of this splicing system (FIG. 3).

The results in FIG. 3 show that with the increase of cisplatin concentration, the inhibitory effect on the growth of Mycobacterium smegmatis containing plasmid PMV261-Kana^{R}-65S-RecA intein was gradually enhanced, and the inhibition rate reached 62% at 20 μM; while the inhibitory effect on the growth of Mycobacterium smegmatis containing the control group Plasmid was moderately inhibited, and the inhibition rate was only 14% at 20 μM. This indicates that cisplatin specifically inhibited the splicing of intein and thus affected the growth of Mycobacterium smegmatis in the kanamycin-resistant medium. That is to say, the screening system in the present invention is feasible and can achieve the specific screening of drugs with inhibitory effects on inteins.

### Example 4

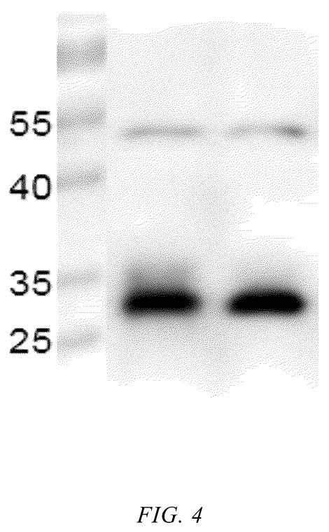

To test the splicing activity of the mini type of RecA intein in Mycobacterium smegmatis, Mycobacterium smegmatis grown in mediums containing 25 μg/mL and 50 μg/mL kanamycin were respectively taken to analyze the splicing activity by Western blot (FIG. 4).

The results in FIG. 4 show that the mini type of RecA intein has a high splicing efficiency at the kanamycin-resistant 65S site when Mycobacterium smegmatis is used as the host, which is up to about 90%, which makes the screening system have a high sensitivity.

The above experimental results show that the screening method constructed in the present invention for Mycobacterium tuberculosis intein inhibitors shows a specific inhibitory effect on inteins with the addition of a cisplatin inhibitor at the kanamycin concentration of 50 μg/mL, fully demonstrating that the screening system of the present invention can achieve the intuitive, quick and convenient screening of substances that have inhibitory effects on the splicing activity of inteins.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A screening system for a Mycobacterium tuberculosis intein splicing inhibitor, wherein: Mycobacterium smegmatis is used as a model bacterium, a gene sequence of a protein regulated by an intein is inserted into a 65S site of a kanamycin-resistant protein gene, a vector with first antibiotic resistance is used for constructing a recombinant plasmid, and the recombinant plasmid is transferred into Mycobacterium smegmatis and is cultured in a medium containing a first antibiotic and kanamycin, to obtain the Mycobacterium tuberculosis intein splicing inhibitor screening system;
the protein regulated by the intein is selected from a full-length or mini-fragment of Mycobacterium tuberculosis RecA intein, DnaB intein, or SufB intein; and the first antibiotic is any antibiotic other than kanamycin.

2. The screening system according to claim 1, wherein the vector is a PMV261 vector.

3. A method for constructing the screening system according to claim 1, comprising steps of:
(1) inserting a gene sequence of a protein regulated by intein into a 65S site of a kanamycin-resistant protein gene to construct a fusion gene;
(2) constructing the fusion gene in a vector with first antibiotic resistance to obtain a recombinant plasmid;
(3) transferring the recombinant plasmid into Mycobacterium smegmatis to obtain recombinant Mycobacterium smegmatis; and
(4) inoculating the recombinant Mycobacterium smegmatis in a medium containing a first antibiotic and kanamycin, to obtain the Mycobacterium tuberculosis intein splicing inhibitor screening system.

4. The method according to claim 3, wherein in the step (2), cleavage sites of the fusion gene and the vector are both BamHI and HindIII.

5. The method according to claim 3, wherein in the step (4), the concentration of kanamycin in the medium is 30-50 μg/mL.

6. The method according to claim 3, wherein in the step (4), the concentration of the first antibiotic in the medium is 40-60 μg/mL.

7. Use of the system according to claim 1 in screening Mycobacterium tuberculosis intein splicing inhibitors, comprising steps of: adding a Mycobacterium tuberculosis intein splicing inhibitor to the screening system, and carrying out high-throughput screening of the Mycobacterium smegmatis intein splicing inhibitor according to the growth of the recombinant Mycobacterium smegmatis.

8. The use according to claim 7, wherein the Mycobacterium tuberculosis intein splicing inhibitor is screened according to an OD value of a bacterial solution of the recombinant Mycobacterium smegmatis.

9. The use according to claim 7, further comprising culturing for 90-110 h after adding a Mycobacterium tuberculosis intein splicing inhibitor to the screening system.

10. Use of the system according to claim 1 in screening anti-tuberculosis drugs.
